(19) United States
(12) Patent Application Publication
(10) Pub. No.: US 2007/0134747 A1
(43) Pub. Date: Jun. 14, 2007

(54) Detection of secreted lipase proteins from Candida species

(75) Inventors: Enrico L. DiGiammarino, Woodstock, GA (US); Kevin P. McGrath, Alpharetta, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/302,991

(22) Filed: Dec. 14, 2005

(51) Int. Cl. G01N 33/53

(52) U.S. Cl. 435/7.92

(57) ABSTRACT

Methods and devices for the detection of proteins secreted by the pathogenic growth form of Candida species are disclosed. The disclosed devices may constitute a method for the diagnosis of acute or chronic infections, including candidiasis, caused by microorganisms of the Candida species, such as C. albicans, for example. The devices of the present invention incorporate antibodies specific to lipase proteins whose expression is differentiated upon the conversion of the Candida species from the commensal to the pathogenic form. The antibodies may be used in assays to allow the diagnosis of candidal infections and disease conditions. Either monoclonal antibodies or polyclonal antibodies may be used, and in the case of the monoclonals, the specific epitopes of the LIP protein may be detected as well as the LIP protein itself.

### BACKGROUND OF THE INVENTION

Candida albicans of the species Candida is the most common fungal pathogen of humans and one of the top five most common microorganisms isolated from blood cultures. Normally, C. albicans is a benign commensal yeast microbe colonizing mucosal surfaces in the mouth and vagina. Under opportune conditions however, C. albicans may become a virulent pathogen able to cause a variety of infections. Depending upon underlying host health and condition, C. albicans may cause infections ranging from vulvovaginal candidiasis to life-threatening disseminated candidiasis that is able to infect virtually every organ of the host.

Virulence of C. albicans is correlated with a change in morphology of the cell from a spherical form to a filamentous hyphal form. In fact, the morphogenic conversion between yeast and hyphal growth forms appears to be critical in the pathogenesis of invasive candidiasis. Virulence and morphogenic conversion are also associated with a change in the cell's transcriptional profile. For example, among the proteins known to display differential expression during the yeast-to-hyphal stage as well as at different stages of infection are members of the lipase family.

Among the problems associated with infection caused by Candida species such as C. albicans is a lack of an accurate diagnostic procedure to recognize the opportunistic form of the cell early on in the disease process. This problem is exacerbated by the fact that there is a broad generality of symptoms for many different infections. For example, among the three most common causes of vaginal infection (vulvovaginal candidiasis, bacterial vaginosis, and trichomoneasis) symptoms may be fairly generic in nature. In addition, concurrent infections may be responsible for symptoms, which may further complicate an accurate diagnosis. In the case of infection due to C. albicans, self-treatment is often possible, but this requires an accurate diagnosis.

What is needed in the art are accurate methods and devices for recognizing the opportunistic form of pathogens such as C. albicans. For instance, a device that could provide a self-diagnosis route for opportunistic C. albicans could be of great benefit to consumers.

### SUMMARY OF THE INVENTION

In one embodiment, the present invention is directed to a diagnostic test kit for detecting a lipase protein within a test sample. The diagnostic test kit may include, for instance, an assay device comprising a fluidic medium. The fluidic medium in turn defines a detection zone within which is immobilized a receptive material. In addition, the detection zone is capable of generating a detection signal that represents the presence or absence of a lipase protein. The diagnostic kit also includes a detection probe conjugated with a binding member. In accordance with the invention, the receptive material, the binding member, or both contain an antibody that specifically binds to the lipase protein.

In another embodiment, the invention is directed to a method for detecting the presence of a lipase protein within a test sample. For example, the method may include contacting an assay device of the invention, for example, an assay device such as that described above, with the test sample and generating a detectable signal at the detection zone that corresponds to the presence or absence of the lipase protein.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a perspective view of one embodiment of a lateral flow assay device of the present invention;

FIG. 2 is a dendrogram displaying the relationship by sequence homology of the C. albicans LIP protein family;

FIG. 3 illustrates the crossreactivity of a polyclonal antibody raised against an LIP5 protein to an LIP4 antigen;

FIG. 4A illustrates the detection by a standard sodium dodecyl (lauryl) sulfate-polyacrylamide gel electrophoresis (SDS-PAGE) of recombinant LIP5 protein;

FIG. 4B illustrates the results of the same samples by western blot when probed with a polyclonal LIP5 antibody;

FIG. 5A illustrates the results of lipase protein detection using the polyclonal antibody raised against an LIP5 protein to probe supernatants from C. albicans cultured over the indicated times; and

FIG. 5B illustrates the results of lipase protein detection using the polyclonal antibody raised against an LIP5 protein to probe cell pellet samples from C. albicans cultured over the indicated times.

### Detailed Description of Representative Embodiments

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

### Definitions

“Polypeptide” refers to a molecular chain of amino acids and does not refer to a specific length of the product. Thus, peptides, oligopeptides and proteins are included within the definition of polypeptide. This term is also intended to include polypeptides that have been subjected to post-expression modifications such as, for example, glycosylations, acetylations, phosphorylations, and so forth.

“Protein” refers to any molecular chain of amino acids that is capable of interacting structurally, enzymatically or otherwise with other proteins, polypeptides or any other organic or inorganic molecule.

“Fragment” refers to an amino acid sequence of a protein or polypeptide that is shorter than the entire protein or polypeptide, but contains at least about 25 consecutive amino acids of the full protein or polypeptide.

“Epitope” refers to a part of a protein that specifically binds an antibody by fitting into the antibody-combining site.

“Test sample” refers to a biological material suspected of containing the analyte. The test sample may be derived from any biological source, such as a physiological fluid, including, blood, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen, and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

“Sequence identity” refers to the subunit sequence similarity between two polymeric molecules. For example, the sequence similarity between two polynucleotides or two polypeptides. When a subunit position in both of the two molecules is occupied by the same monomeric subunit, then they are identical at that position. The identity between two sequences is a direct function of the number of matching or identical positions. For example, if half of the positions in two peptide or compound sequences are identical, then the two sequences are 50% identical. The identity between two sequences is a direct function of the number of matching or identical positions. Thus, if a portion of the reference sequence is deleted in a particular peptide, that deleted section is not counted for purposes of calculating sequence identity. For example, when comparing a first polymer including monomers R₁R₂R₃R₄R₅R₆ with another polymer including monomers R₁R₂R₃R₄R₆, the two polymers have 5 out of 6 positions in common, and therefore would be described as sharing 83.3% sequence identity.

### DETAILED DESCRIPTION

In general, the present invention is directed to methods and devices for accurately determining the presence of proteins the expression of which is differentiated upon transformation of a Candida species from the commensal to the pathogenic form. More particularly, the presently disclosed methods and devices may be utilized to accurately detect the presence of members of the lipase (LIP) family of proteins in a sample via recognition and binding by antibodies specific to the LIP proteins. Accordingly, one embodiment of the present invention is directed to the diagnosis of a yeast infection via recognition of LIP proteins, for instance for diagnosis of vulvovaginal candidiasis caused by opportunistic C. albicans.

The invention is not limited to this embodiment, however, and in other embodiments the methods and devices disclosed herein may be beneficially utilized for the detection and diagnosis of other diseases caused by the opportunistic infection of C. albicans, such as disseminated candidiasis.

Moreover, C. albicans is not the only Candida species that is known to possess LIP genes. For example, C. parapsilosis, C. tropicalis, and C. krusei are believed to possess LIP genes (see, Fu, et al., ‘Cloning and characterization of a gene (LIP1) which encodes a lipase from the pathogenic yeast Candida albicans,’ Microbiology, Vol. 143, p. 331-340 (1997)). As such, in other embodiments, the methods and devices of the present invention may be beneficially utilized in diagnosis of disease caused by other pathogens in which the disease state is characterized by differential LIP protein expression, and in one particular embodiment, for diagnosis of disease caused by other Candida pathogens, in addition to the pathogenic C. albicans to which the following discussion is primarily directed.

The methods and devices of the present invention utilize antibodies specific to LIP proteins to provide a route for the detection of secreted Candidal antigens present in a biological fluid (e.g. blood, serum, plasma, saliva, urine, cerebrospinal fluid, genitourinary tract) or other biological material (e.g., tissues, bone, muscle, cartilage, or skin). Accordingly, the present invention may constitute a method for the diagnosis of acute or chronic infections, including candidiasis caused by pathogens of the Candida species.

The Assay Devices

The devices of the present invention perform heterogeneous immunoassays that incorporate antibodies specific to LIP proteins to allow the detection of the pathogenic form of Candida species. A heterogeneous assay is an assay in which uncomplexed labeled species are separated from complexed labeled species. Separation may be carried out by physical separation, e.g., by transferring one of the species to another reaction vessel, filtration, centrifugation, chromatography, solid phase capture, magnetic separation, and so forth, and may include one or more washing steps. The separation may also be nonphysical in that no transfer of one or both of the species is conducted, but the species are separated from one another in situ. Heterogeneous immunoassays of the invention utilize the mechanisms of the immune systems, i.e., antibodies that are produced in response to the presence of antigens that are pathogenic or foreign to a host organism. These immunoreactants are capable of binding with one another, thereby causing a highly specific reaction mechanism that may be used to determine the presence or concentration of that particular antigen (e.g., LIP protein) in a fluid test sample.

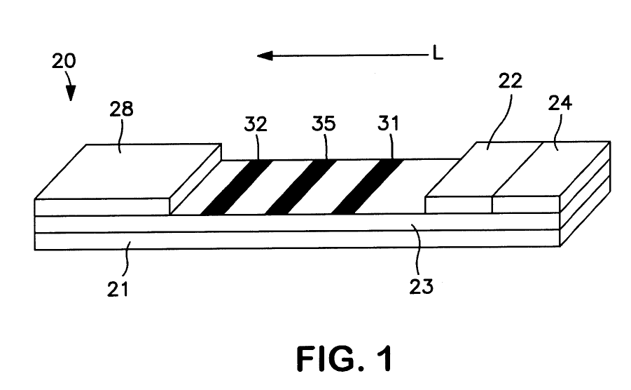

In one preferred embodiment, the assay device of the present invention is a lateral flow assay device. Referring now to FIG. 1, one embodiment of a lateral flow assay device 20 of the present invention will now be described in more detail. As shown, the device 20 contains a porous membrane 23 that acts as a fluidic medium and is optionally supported by a rigid material 21. The fluidic medium of the disclosed lateral flow assay devices is not limited to a porous membrane, however. For instance, in addition to flow through devices that utilize a porous membrane as a fluidic medium, assay devices that utilize one or more fluidic channels, or any other suitable component, as a fluidic medium are also encompassed by the present invention.

In general, the porous membrane 23 may be made from any of a variety of materials through which the test sample is capable of passing. For example, the materials used to form the porous membrane 23 may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the porous membrane 23 is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The size and shape of the porous membrane 23 may generally vary as is readily recognized by those skilled in the art. For instance, a porous membrane strip may have a length of from about 10 to about 100 millimeters, in some embodiments from about 20 to about 80 millimeters, and in some embodiments, from about 40 to about 60 millimeters. The width of the membrane strip may range from about 0.5 to about 20 millimeters, in some embodiments from about 1 to about 15 millimeters, and in some embodiments, from about 2 to about 10 millimeters. In one embodiment, the thickness of the membrane strip may be small enough to allow transmission-based detection. For example, the membrane strip may have a thickness less than about 500 micrometers, in some embodiments less than about 250 micrometers, and in some embodiments, less than about 150 micrometers.

As stated above, the support 21 carries the porous membrane 23. For example, the support 21 may be positioned directly adjacent to the porous membrane 23 as shown in FIG. 1, or one or more intervening layers may be positioned between the porous membrane 23 and the support 21. Regardless, the support 21 may generally be formed from any material able to carry the porous membrane 23. The support 21 may be formed from a material that is transmissive to light, such as transparent or optically diffuse (e.g., translucent) materials. Also, it is generally desired that the support 21 is liquid-impermeable so that fluid flowing through the membrane 23 does not leak through the support 21. Examples of suitable materials for the support include, but are not limited to, glass; polymeric materials, such as polystyrene, polypropylene, polyester (e.g., Mylar® film), polybutadiene, polyvinylchloride, polyamide, polycarbonate, epoxides, methacrylates, and polymelamine; and so forth. To provide a sufficient structural backing for the porous membrane 23, the support 21 is generally selected to have a certain minimum thickness. For example, the support 21 may have a thickness that ranges from about 100 to about 5,000 micrometers, in some embodiments from about 150 to about 2,000 micrometers, and in some embodiments, from about 250 to about 1,000 micrometers. For instance, one suitable membrane strip suitable for use as a support having a thickness of about 125 micrometers may be obtained from Millipore Corp. of Bedford, Mass. under the name “SHF180UB25.”

As is well known the art, the porous membrane 23 may be cast onto the support 21, wherein the resulting laminate may be die-cut to the desired size and shape. Alternatively, the porous membrane 23 may simply be laminated to the support 21 with, for example, an adhesive. In some embodiments, a nitrocellulose or nylon porous membrane is adhered to a Mylar® film. An adhesive is used to bind the porous membrane to the Mylar® film, such as a pressure-sensitive adhesive. Laminate structures of this type are believed to be commercially available from Millipore Corp. of Bedford, Mass. Still other examples of suitable laminate assay device structures are described in U.S. Pat. No. 5,075,077 to Durley, III, et al., which is incorporated herein in its entirety by reference thereto for all purposes.

The device 20 may also contain an absorbent pad 28. The absorbent pad 28 generally receives fluid that has migrated through the entire porous membrane 23. As is well known in the art, the absorbent pad 28 may assist in promoting capillary action and fluid flow through the membrane 23.

To initiate the detection of an analyte within the test sample, a user may directly apply the test sample to a portion of the porous membrane 23 through which it may then travel in the direction illustrated by arrow “L” in FIG. 1. Alternatively, the test sample may first be applied to a sample pad 24 that is in fluid communication with the porous membrane 23. Some suitable materials that may be used to form the sample pad 24 include, but are not limited to, nitrocellulose, cellulose, porous polyethylene pads, and glass fiber filter paper. If desired, the sample pad 24 may also contain one or more assay pretreatment reagents, either diffusively or non-diffusively attached thereto. For example, in one embodiment, a calibration analyte may be disposed on the sample pad 24 so that it contacts the test sample upon application thereto.

In the illustrated embodiment, the test sample travels from the sample pad 24 to a conjugate pad 22 that is placed in communication with one end of the sample pad 24. The conjugate pad 22 is formed from a material through which the test sample is capable of passing. For example, in one embodiment, the conjugate pad 22 is formed from glass fibers. Although only one conjugate pad 22 is shown, it should be understood that multiple conjugate pads may also be used in the present invention. In one particular embodiment of the present invention, detection and optionally calibration probes (not shown) may be applied to the conjugate pad 22. After application, the probes are then dried to inhibit migration therefrom. The conjugate pad 22 provides a matrix for the deposition of the probes so that they are free to migrate when rehydrated. More specifically, when a liquid test sample contacts the probes, they are rehydrated and become re-suspended and/or re-solubilized. Of course, it should be understood that the probes may be applied to various other locations of the assay device 20 as well, such as directly to the membrane 23, so long as they are capable of being rehydrated by the test sample upon contact therewith.

To facilitate the detection of the LIP protein within a test sample, a detectable substance may be pre-applied to the sample pad and/or conjugate pad, or previously mixed with a diluent or test sample. The detectable substance may function as a detection probe that is detectable either visually or by an instrumental device. Any substance generally capable of producing a signal that is detectable visually or by an instrumental device may be used as detection probes. Suitable detectable substances may include, for instance, luminescent compounds (e.g., fluorescent, phosphorescent, etc.); radioactive compounds; visual compounds (e.g., colored dye or metallic substance, such as gold); liposomes or other vesicles containing signal-producing substances; enzymes and/or substrates, and so forth. Other suitable detectable substances may be described in U.S. Pat. Nos. 5,670,381 to Jou, et al. and 5,252,459 to Tarcha, et al., which are incorporated herein in their entirety by reference thereto for all purposes. If the detectable substance is colored, the ideal electromagnetic radiation is light of a complementary wavelength. For instance, blue detection probes strongly absorb red light.

In some embodiments, the detectable substance may be a luminescent compound that produces an optically detectable signal. For example, suitable fluorescent molecules may include, but are not limited to, fluorescein, europium chelates, phycobiliprotein, phycoerythrin, phycocyanin, allophycocyanin, o-phthaldehyde, fluorescamine, rhodamine, and their derivatives and analogs. Other suitable fluorescent compounds are semiconductor nanocrystals commonly referred to as “quantum dots.” For example, such nanocrystals may contain a core of the formula CdX, wherein X is Se, Te, S, and so forth. The nanocrystals may also be passivated with an overlying shell of the formula YZ, wherein Y is Cd or Zn, and Z is S or Se. Other examples of suitable semiconductor nanocrystals may also be described in U.S. Pat. Nos. 6,261,779 to Barbera-Guillem, et al. and 6,585,939 to Dapprich, which are incorporated herein in their entirety by reference thereto for all purposes.

Further, suitable phosphorescent compounds may include metal complexes of one or more metals, such as ruthenium, osmium, rhenium, iridium, rhodium, platinum, indium, palladium, molybdenum, technetium, copper, iron, chromium, tungsten, zinc, and so forth. Especially preferred are ruthenium, rhenium, osmium, platinum, and palladium. The metal complex may contain one or more ligands that facilitate the solubility of the complex in an aqueous or non-aqueous environment. For example, some suitable examples of ligands include, but are not limited to, pyridine; pyrazine; isonicotinamide; imidazole; bipyridine; terpyridine; phenanthroline; dipyridophenazine; porphyrin; porphine; and derivatives thereof. Such ligands may be, for instance, substituted with alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxy-succinimide.

Porphyrins and porphine metal complexes possess pyrrole groups coupled together with methylene bridges to form cyclic structures with metal chelating inner cavities. Many of these molecules exhibit strong phosphorescence properties at room temperature in suitable solvents (e.g., water) and an oxygen-free environment. Some suitable porphyrin complexes that are capable of exhibiting phosphorescent properties include, but are not limited to, platinum (II) coproporphyrin-I and III, palladium (II) coproporphyrin, ruthenium coproporphyrin, zinc(II)-coproporphyrin-I, derivatives thereof, and so forth. Similarly, some suitable porphine complexes that are capable of exhibiting phosphorescent properties include, but not limited to, platinum(II) tetra-meso-fluorophenylporphine and palladium(II) tetra-meso-fluorophenylporphine. Still other suitable porphyrin and/or porphine complexes are described in U.S. Pat. Nos. 4,614,723 to Schmidt, et al.; 5,464,741 to Hendrix; 5,518,883 to Soini; 5,922,537 to Ewart, et al.; 6,004,530 to Sagner, et al.; and 6,582,930 to Ponomarev, et al., which are incorporated herein in their entirety by reference thereto for all purposes.

Bipyridine metal complexes may also be utilized as phosphorescent compounds. Some examples of suitable bipyridine complexes include, but are not limited to, bis[(4,4′-carbomethoxy)-2,2′-bipyridine]2-[3-(4-methyl-2,2′-bipyridine-4-yl)propyl]-1,3-dioxolane ruthenium (II); bis(2,2′bipyridine)[4-(butan-1-al)-4′-methyl-2,2′-bi-pyridine]ruthenium (II); bis(2,2′-bipyridine)[4-(4′-methyl-2,2′-bipyridine-4′-yl)-butyric acid]ruthenium (II); tris(2,2′bipyridine)ruthenium (II); (2,2′-bipyridine) [bis-bis(1,2-diphenylphosphino)ethylene]2-[3-(4-methyl-2,2′-bipyridine-4′-yl)propyl]-1,3-dioxolane osmium (II); bis(2,2′-bipyridine)[4-(4′-methyl-2,2′-bipyridine)-butylamine]ruthenium (II); bis(2,2′-bipyridine)[1-bromo-4(4′-methyl-2,2′-bipyridine-4-yl)butane]ruthenium (II); bis(2,2′-bipyridine)maleimidohexanoic acid, 4-methyl-2,2′-bipyridine-4′-butylamide ruthenium (II), and so forth. Still other suitable metal complexes that may exhibit phosphorescent properties may be described in U.S. Pat. Nos. 6,613,583 to Richter, et al.; 6,468,741 to Massey, et al.; 6,444,423 to Meade, et al.; 6,362,011 to Massey, et al.; 5,731,147 to Bard, et al.; and 5,591,581 to Massey, et al., which are incorporated herein in their entirety by reference thereto for all purposes.

In some cases, luminescent compounds may have a relatively long emission lifetime and/or may have a relatively large “Stokes shift.” The term “Stokes shift” is generally defined as the displacement of spectral lines or bands of luminescent radiation to a longer emission wavelength than the excitation lines or bands. A relatively large Stokes shift allows the excitation wavelength of a luminescent compound to remain far apart from its emission wavelengths and is desirable because a large difference between excitation and emission wavelengths makes it easier to eliminate the reflected excitation radiation from the emitted signal. Further, a large Stokes shift also minimizes interference from luminescent molecules in the sample and/or light scattering due to proteins or colloids, which are present with some body fluids (e.g., blood). In addition, a large Stokes shift also minimizes the requirement for expensive, high-precision filters to eliminate background interference. For example, in some embodiments, the luminescent compounds have a Stokes shift of greater than about 50 nanometers, in some embodiments greater than about 100 nanometers, and in some embodiments, from about 100 to about 350 nanometers.

For example, exemplary fluorescent compounds having a large Stokes shift include lanthanide chelates of samarium (Sm (III)), dysprosium (Dy (III)), europium (Eu (III)), and terbium (Tb (III)). Such chelates may exhibit strongly red-shifted, narrow-band, long-lived emission after excitation of the chelate at substantially shorter wavelengths. Typically, the chelate possesses a strong ultraviolet excitation band due to a chromophore located close to the lanthanide in the molecule. Subsequent to excitation by the chromophore, the excitation energy may be transferred from the excited chromophore to the lanthanide. This is followed by a fluorescence emission characteristic of the lanthanide. Europium chelates, for instance, have Stokes shifts of about 250 to about 350 nanometers, as compared to only about 28 nanometers for fluorescein. Also, the fluorescence of europium chelates is long-lived, with lifetimes of about 100 to about 1000 microseconds, as compared to about 1 to about 100 nanoseconds for other fluorescent labels. In addition, these chelates have narrow emission spectra, typically having bandwidths less than about 10 nanometers at about 50% emission. One suitable europium chelate is N-(p-isothiocyanatobenzyl)-diethylene triamine tetraacetic acid-Eu⁺³.

In addition, lanthanide chelates that are inert, stable, and intrinsically fluorescent in aqueous solutions or suspensions may also be used in the present invention to negate the need for micelle-forming reagents, which are often used to protect chelates having limited solubility and quenching problems in aqueous solutions or suspensions. One example of such a chelate is 4-[2-(4-isothiocyanatophenyl)ethynyl]-2,6-bis([N,N-bis(carboxymethyl)amino]methyl)-pyridine [Ref: Lovgren, T., et al.; Clin. Chem. 42, 1196-1201 (1996)]. Several lanthanide chelates also show exceptionally high signal-to-noise ratios. For example, one such chelate is a tetradentate β-diketonate-europium chelate [Ref: Yuan, J. and Matsumoto, K.; Anal. Chem. 70, 596-601 (1998)]. In addition to the fluorescent labels described above, other labels that are suitable for use in the present invention may be described in U.S. Pat. Nos. 6,030,840 to Mullinax, et al.; 5,585,279 to Davidson; 5,573,909 to Singer, et al.; 6,242,268 to Wieder, et al.; and 5,637,509 to Hemmila, et al., which are incorporated herein in their entirety by reference thereto for all purposes.

Detectable substances, such as described above, may be used alone or in conjunction with a particle (sometimes referred to as “beads” or “microbeads”). For instance, naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., may be used. Further, synthetic particles may also be utilized. For example, in one embodiment, latex microparticles that are labeled with a fluorescent or colored dye are utilized. Although any synthetic particle may be used in the present invention, the particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable particles may be described in U.S. Pat. Nos. 5,670,381 to Jou, et al.; 5,252,459 to Tarcha, et al.; and U.S. Patent Publication No. 2003/0139886 to Bodzin, et al., which are incorporated herein in their entirety by reference thereto for all purposes. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc. Metallic particles (e.g., gold particles) may also be utilized in the present invention.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 100 microns, in some embodiments, from about 1 nanometer to about 10 microns, and in some embodiments, from about 10 to about 100 nanometers.

In some instances, it may be desired to modify the detection probes so that they are more readily able to bind to the analyte. In such instances, the detection probes may be modified with certain specific binding members that are adhered thereto to form conjugated probes. For instance, the detection probe may be conjugated with antibodies as are further described below that are specific to LIP proteins. The detection probe antibody may be a monoclonal or polyclonal antibody or a mixture(s) or fragment(s) thereof.

The antibodies may generally be attached to the detection probes using any of a variety of well-known techniques. For instance, covalent attachment of the antibodies to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer as the surface of the detection probe may contain a relatively high surface concentration of polar groups. In addition, although detection probes are often functionalized after synthesis, such as with poly(thiophenol), the detection probes may be capable of direct covalent linking with an antibody without the need for further modification. For example, in one embodiment, the first step of conjugation is activation of carboxylic groups on the probe surface using carbodiimide. In the second step, the activated carboxylic acid groups are reacted with an amino group of an antibody to form an amide bond. The activation and/or antibody coupling may occur in a buffer, such as phosphate-buffered saline (PBS) (e.g., pH of 7.2) or 2-(N-morpholino) ethane sulfonic acid (MES) (e.g., pH of 5.3). The resulting detection probes may then be contacted with ethanolamine, for instance, to block any remaining activated sites. Overall, this process forms a conjugated detection probe, where the antibody is covalently attached to the probe. Besides covalent bonding, other attachment techniques, such as physical adsorption, may also be utilized in the present invention.

In one embodiment, the antibody may be detectably labeled by linking to an enzyme. The enzyme, in turn, when later exposed to a substrate, will react with the substrate in such a manner as to produce a chemical moiety which may be detected as, for example, by spectrophotometric or fluorometric means. Examples of enzymes which may be used to detectably label the antibodies as herein described include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase.

Another technique that may also result in greater sensitivity when used in conjunction with the present invention consists of coupling the antibodies to low molecular weight haptens. The haptens may then be specifically detected by means of a second reaction. For example, it is common to use such haptens as biotin (reacting with avidin) or dinitrophenol, pyridoxal and fluorescamine (reacting with specific antihapten antibodies) in this manner.

The antibodies of the present invention also may be detectably labeled by coupling to a chemiluminescent compound. The presence of the chemiluminescent-tagged antibody is then determined by detecting the presence of luminescence that arises during the course of the chemical reaction. Examples of particularly useful chemiluminescent labeling compounds are luminol, isoluminol, theromatic acridinium ester, imidazole, acridinium salt and oxalate ester. Likewise, a bioluminescent compound may be used to label the antibodies as further described below. Bioluminescence is a type of chemiluminescence found in biological systems in which a catalytic protein increases the efficiency of the chemiluminescent reaction. The presence of a bioluminescent reagent is determined by detecting the presence of luminescence. Important bioluminescent compounds for purposes of labeling are luciferin, luciferase and aequorin.

Referring again to FIG. 1, the porous membrane 23 also defines various zones configured to perform the assay. For instance, the porous membrane 23 defines a detection zone 31 that contains a receptive material that is capable of binding to the conjugated detection probes (or complexes thereof) that pass through the length of the membrane 23. The receptive material is immobilized on the porous membrane 23 and may be an antibody that is the same or different from the antibody of the conjugated detection probe. In one embodiment, the two antibodies may be different. For example, the receptive material may include a first monoclonal antibody specific to a first epitope of an LIP protein, and the antibody of the conjugated detection probe may be a second, different monoclonal antibody specific to a second epitope of the LIP protein. In sandwich assay formats, for example, the receptive material may serve as a stationary binding site for complexes formed between the LIP proteins in the test sample and the conjugated detection probes. Specifically, the LIP proteins have two or more binding sites (e.g., epitopes). Upon reaching the detection zone 31, one of these binding sites is occupied by the antibody of the conjugated probe. However, a free binding site of the LIP protein may bind to the immobilized first receptive material. Upon being bound to the immobilized receptive material, the complexed probes form a new ternary sandwich complex.

Other than the detection zone 31, the lateral flow device 20 may also define various other zones for enhancing detection accuracy. For example, in embodiments in which high analyte concentrations are a concern, the assay device 20 may contain an indicator zone 35 that is positioned downstream from the detection zone 31 and is configured to provide information as to whether the analyte concentration has reached the saturation concentration (“hook effect” region) for the assay. The indicator zone 35 contains a second receptive material that is immobilized on the membrane 23 and serves as a stationary binding site for the conjugated detection probes. To accomplish the desired binding within the indicator zone 35, it is generally desired that the second receptive material is capable of differentiating between those detection probes that are complexed with the LIP proteins and those that remain uncomplexed. For example, in one embodiment, the second receptive material includes a molecule that has at least one epitope in common with the LIP proteins, such as entire protein molecules, or derivatives or fragments (e.g., analogs) thereof, so that it is capable of specifically binding to an antibody conjugate when it is uncomplexed with the LIP proteins in the sample.

Although the detection zone 31 and optional indicator zone 35 may provide accurate results, it is sometimes difficult to determine the relative concentration of the analyte within the test sample under actual test conditions. Thus, the assay device 20 may include a calibration zone 32. In this embodiment, the calibration zone 32 is formed on the membrane 23 and is positioned downstream from the detection zone 31 and optional indicator zone 35. Alternatively, however, the calibration zone 32 may also be positioned upstream from the detection zone 31 and/or optional indicator zone 35. The calibration zone 32 is provided with a third receptive material that is capable of binding to any calibration probes that pass through the length of the membrane 23. When utilized, the calibration probes may contain a detectable substance that is the same or different than the detectable substance used for the detection probes. Moreover, the calibration probes may also be conjugated with a specific binding member for the LIP proteins. For example, in one embodiment, biotinylated calibration probes may be used. Generally speaking, the calibration probes are selected in such a manner that they do not bind to the first or second receptive material at the detection zone 31 and indicator zone 35. The third receptive material of the calibration zone 32 may be the same or different than the receptive materials used in the detection zone 31 or indicator zone 35. For example, in one embodiment, the third receptive material is a third antibody specific to the LIP proteins. It may also be desired to utilize various non-biological materials for the third receptive material (e.g., polyelectrolytes) of the calibration zone 32, such as described in U.S. Patent Application Publication No. 2003/0124739 to Song, et al., which is incorporated herein in its entirety by reference thereto for all purposes.

The detection zone 31, indicator zone 35, and calibration zone 32 may each provide any number of distinct detection regions so that a user may better determine the concentration of one or more LIP proteins within a test sample. Each region may contain the same receptive materials, or may contain different receptive materials. For example, the zones may include two or more distinct regions (e.g., lines, dots, etc.). The regions may be disposed in the form of lines in a direction that is substantially perpendicular to the flow of the test sample through the assay device 20. Likewise, in some embodiments, the regions may be disposed in the form of lines in a direction that is substantially parallel to the flow of the test sample through the assay device 20.

In some cases, the membrane 23 may also define a control zone (not shown) that gives a signal to the user that the assay is performing properly. For instance, the control zone (not shown) may contain an immobilized receptive material that is generally capable of forming a chemical and/or physical bond directly with the detection probes or with the receptive material antibody immobilized on the probes. In addition, it may also be desired to utilize various non-biological materials for the control zone receptive material. For instance, in some embodiments, the control zone receptive material may also include a polyelectrolyte, such as described above, that may bind to uncaptured probes. Because the receptive material at the control zone is only specific for probes, a signal forms regardless of whether the analyte is present. The control zone may be positioned at any location along the membrane 23, but is preferably positioned downstream from the detection zone 31 and the indicator zone 35.

Qualitative, semi-quantitative, and quantitative results may be obtained in accordance with the present invention. For example, when it is desired to semi-quantitatively or quantitatively detect an analyte, the intensity of any signals produced at the detection zone 31, indicator zone 35, and/or calibration zone 32 may be measured with an optical reader. The actual configuration and structure of the optical reader may generally vary as is readily understood by those skilled in the art. For example, optical detection techniques that may be utilized include, but are not limited to, luminescence (e.g., fluorescence, phosphorescence, etc.), absorbance (e.g., fluorescent or non-fluorescent), diffraction, etc. One suitable reflectance spectrophotometer is described, for instance, in U.S. Patent App. Pub. No. 2003/0119202 to Kaylor, et al., which is incorporated herein in its entirety by reference thereto for all purposes. In another embodiment, a reflectance-mode spectrofluorometer may be used to detect the intensity of a fluorescence signal. Suitable spectrofluorometers and related detection techniques are described, for instance, in U.S. Patent App. Pub. No. 2004/0043502 to Song, et al., which is incorporated herein in its entirety by reference thereto for all purposes. Likewise, a transmission-mode detection system may also be used to signal intensity.

Detection and calibration may be performed automatically and/or manually in accordance with the present invention. For example, a microprocessor may optionally be employed to convert signal intensities from a detector to a result that quantitatively or semi-quantitatively indicates the concentration of the LIP protein in the sample. The microprocessor may include memory capability to allow the user to recall the last several results. Those skilled in the art will appreciate that any suitable computer-readable memory devices, such as RAM, ROM, EPROM, EEPROM, flash memory cards, digital video disks, Bernoulli cartridges, and so forth, may be used. If desired, the results may be conveyed to a user using a liquid crystal (LCD) or LED display.

Although various embodiments of device configurations have been described above, it should be understood, that a device of the present invention may generally have any configuration desired, and need not contain all of the components described above. Various other device configurations, for instance, are described in U.S. Pat. Nos. 5,395,754 to Lambotte, et al.; 5,670,381 to Jou, et al.; and 6,194,220 to Malick, et al., which are incorporated herein in their entirety by reference thereto for all purposes.

Various formats may be used to test for the presence or absence of an LIP protein using the assay devices of the present invention. For instance, a “sandwich” format typically involves mixing the test sample with detection probes conjugated with a specific binding member (e.g., antibody) for the analyte to form complexes between the analyte and the conjugated probes. These complexes are then allowed to contact a receptive material (e.g., antibodies) immobilized within the detection zone. Binding occurs between the analyte/probe conjugate complexes and the immobilized receptive material, thereby localizing “sandwich” complexes that are detectable to indicate the presence of the analyte. This technique may be used to obtain quantitative or semi-quantitative results. Some examples of such sandwich-type assays are described by U.S. Pat. Nos. 4,168,146 to Grubb, et al. and 4,366,241 to Tom, et al., which are incorporated herein in their entirety by reference thereto for all purposes. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule. Examples of competitive immunoassay devices are described in U.S. Pat. Nos. 4,235,601 to Deutsch, et al., 4,442,204 to Liotta, and 5,208,535 to Buechler, et al., which are incorporated herein in their entirety by reference thereto for all purposes. Various other device configurations and/or assay formats are also described in U.S. Pat. Nos. 5,395,754 to Lambotte, et al.; 5,670,381 to Jou, et al.; and 6,194,220 to Malick, et al., which are incorporated herein in their entirety by reference thereto for all purposes.

Although various assay device configuration have been described herein, it should be understood that any known assay device may be utilized that is capable of incorporating an antibody in accordance with the present invention. For example, electrochemical affinity assay devices may also be utilized, which detect an electrochemical reaction between an LIP protein (or complex thereof) and a capture ligand on an electrode strip. For example, various electrochemical assays and assay devices are described in U.S. Pat. Nos. 5,508,171 to Walling, et al.; 5,534,132 to Vreeke, et al.; 6,241,863 to Monbouguette; 6,270,637 to Crismore, et al.; 6,281,006 to Heller, et al.; and 6,461,496 to Feldman, et al., which are incorporated herein in their entirety by reference thereto for all purposes.

Diagnostic Kits

The present invention is also directed to kits for performing the disclosed assays. The kits may include antibodies to LIP proteins and/or fragments thereof as described herein (raised against whole LIP proteins or active immunoreactive fragments or analogs thereof) which may be optionally immobilized, as well as any necessary reagents and equipment to prepare the biological sample for and to conduct analysis, e.g. preservatives, reaction media such as nontoxic buffers, microtiter plates, micropipettes, etc. The antibodies may be lyophilized or cryopreserved. The types of immunoassays that may be incorporated in kit form are many. In one embodiment, the antibodies of the invention as set forth herein may be used in kits to provide a method for self-diagnosis of a candidal infection. Such diagnostic kits may be prepared so as to be suitable for determining the presence of LIP proteins that will bind to the antibodies of the invention. These diagnostic kits will generally include the antibodies as herein described along with suitable means for detecting binding by that antibody such as would be readily understood by one skilled in this art. For example, the means for detecting binding of the antibody may comprise a detectable label that is linked to the antibody as described above. These kits may then be used in diagnostic methods to detect the presence of a candidal infection in a sample through determination of whether the antibodies bind to analytes in the sample, which would indicated the presence of such microorganisms either in the sample itself or in the source.

In one particular embodiment, a kit of the present invention may be useful in methods of monitoring the level of Candidal antibodies or antigens in a test sample obtained from a human or animal. For example, the kit may be useful in monitoring the level of Candidal antibodies or antigens in vaginal fluid, whole blood or serum, saliva, urine, skin or the like. If monitoring the level of Candidal antigen is desired, the kit may include a Candidal antibody in accordance with the present invention as described herein along with a means of determining the level of binding to that antibody. When it is desired to measure the level of Candidal antibodies in a sample, the kit will preferably include an isolated Candidal epitopic carbohydrate moietyprotein, or peptide such as described herein, e.g., a protein or peptide selected from the group consisting of the LIP proteins along with means for detecting binding of those antigens to Candidal antibodies present in the sample.

The Antibodies

The antibodies of the disclosed devices may be monoclonal or polyclonal and may be generated using any suitable method as is known to one of skill in the art. For example, in one embodiment, one or more isolated and/or purified or recombinantly produced LIP proteins may be utilized to generate the antibodies.

A family of 10 LIP genes encodes the LIP proteins expressed by C. albicans. Members of the protein family have been found to be expressed differentially in different tissues and at different stages of infection. For instance, transcripts of most lipase genes have been detected during the yeast-to-hyphal transition, while LIP5, LIP6, LIP8, and LIP9 have been found to be expressed during experimental infection of mice (see, Hube, et al., ‘Secreted lipases of Candida albicans: cloning, characterization and expression analysis of a new gene family with at least ten members.’ Arch. Microbiol., Vol. 174, No. 5, p. 362-74 (2000)). A large volume of information concerning these proteins as well as their encoding genes is generally known to one of ordinary skill in the art and available from many sources such as, for example, the Candida genome database (www.candidagenome.org). Names and identifiers of these 10 LIP genes are summarized below in Table 1.

TABLE 1

| | | | GENBANK |
|---|---|---|---|
| STANDARD | SYSTEMATIC | | ACCESSION |
| NAME | NAME | ALIAS | NUMBER |
| LIP1 | orf19.4821 | orf19.12284, IPF22431.1, | AF188894 |
| | | IPF14712.2, Contig4-2746_0012, | |
| | | Contig4-2746_0014, orf6.2398 | |
| LIP2 | orf19.4804 | orf19.12267, IPF22428.1, | AF189152 |
| | | IPF6120.2, Contig4-3040_0021, | |
| | | orf6.5210 | |
| LIP3 | orf19.4856 | orf19.12319, IPF22432.1, | AF191316 |
| | | IPF7736.2, Contig4-2920_0010, | |
| | | orf6.7355 | |
| LIP4 | orf19.2133 | orf19.9680, IPF19830.2, | AF191317 |
| | | IPF15657.2, Contig4-2687_0005, | |
| | | orf6.288, orf6.5367 | |
| LIP5 | orf19.5179 | orf19.12646, IPF22057.1, | AF191318 |
| | | IPF5395.2, Contig4-1845_0002, | |
| | | orf6.6957, LIP2-3 | |
| LIP6 | orf19.4823 | orf19.12286, IPF22433.1, | AF191319 |
| | | IPF11788.2, Contig4-2746_0007, | |
| | | orf6.7388 | |
| LIP7 | orf19.7320 | IPF1964.2, Contig4-2511_0006, | AF191320 |
| | | orf6.8401 | |
| LIP8 | orf19.1345 | orf19.8925, orf6.2643, IPF7914.2, | AF191321 |
| | | IPF29487.1, Contig4-3068_0044 | |
| LIP9 | orf19.5172 | orf19.12639, IPF22071.1, IPF22065.1, | AF191322 |
| | | IPF5407.2, IPF5406.2, | |
| | | Contig4-3092_0045, orf6.6964 | |
| LIP10 | orf19.4822 | orf19.12285, IPF22423.1, IPF11790.1, | AF191323 |
| | | Contig4-2746_0010, orf6.7389 | |

Elevated levels of LIP5, LIP8, and LIP4 mRNA transcripts have been reported to be associated with C. albicans infections (Stehr, F., et al., ‘Expression analysis of the Candida albicans lipase gene family during experimental infections and in patient samples.’ FEMS Yeast Research 4, pp. 401-408 (2004)). Accordingly, in one preferred embodiment, the present invention is directed to methods and devices for detecting these particular proteins as biomarkers for the detection of candidosis.

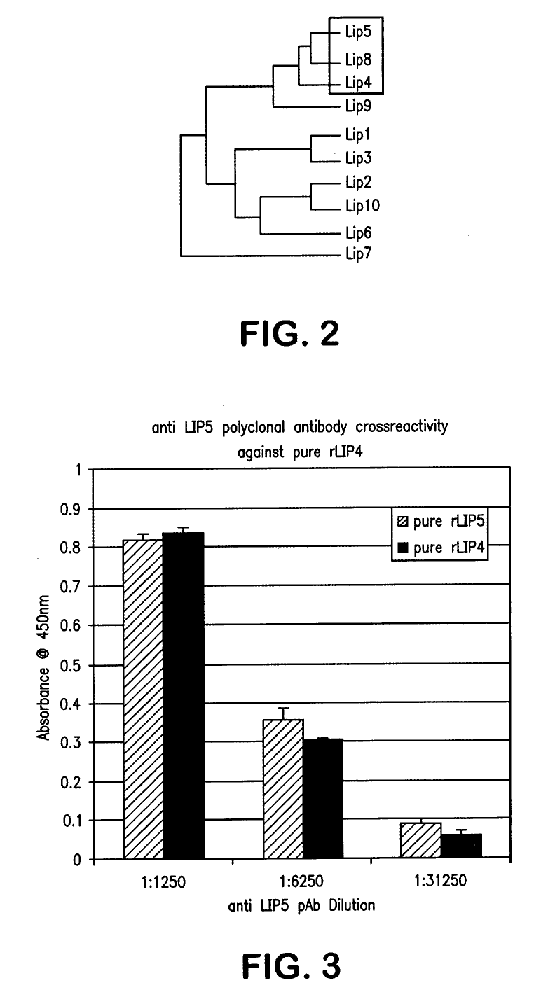

FIG. 2 is a dendrogram displaying the relationship by sequence homology of the C. albicans LIP protein family. Members of this lipase family are quite similar with respect to sequences. For instance, among the family, amino acid sequence identities range from 33% to 80%. As may be seen with reference to the Figure, the LIP5 protein (Lip5), the LIP8 protein (Lip8), and the LIP4 protein (Lip4) are closely related. In fact, these three proteins have more than 73% sequence identity (Hube, B., et al., 2000). For instance, when considering the native proteins, sequence identities calculated using the pairwise blast tool (blastp) available from the National Center for Biotechnology Information (http://www.ncbi.nlm.nih.gov/blast/bl2seq/wblast2.cgi) are determined to be 75% between LIP5 protein and LIP4 protein, 80% between LIP5 protein and LIP8 protein, and 76% between LIP4 protein and LIP8 protein.

According to the present invention, it has been found that polyclonal antibodies raised against one of these proteins may cross-react among the others. As such, in one embodiment, an assay device of the invention may include an antibody raised against one of these proteins, and may beneficially be utilized to detect other antigens of the family, in addition to that specific protein.

In one embodiment, nucleic acids encoding one or more of the LIP proteins or immunogenic epitopes thereof may be expressed and purified to obtain suitable quantity of protein that may then be utilized to generate the antibodies of the disclosed invention. For instance, recombinant expression of an LIP protein or targeted segment thereof may include amplifying a targeted nucleotide sequence encoding the polypeptide from genomic DNA obtained from a C. albicans culture and then introducing the nucleotide sequence into an expression vector adapted for use in the desired expression system. The nucleotide sequence of such a construct is not limited to cDNA sequences, however, and the LIP protein-encoding construct may include variations as are known to those of skill in the art including orthologs, homologs, and alleles of the cDNA encoding the LIP proteins, provided the transcribed protein product may exhibit the same or superior immunogenic response in a host as the cDNA encoded transcription products.

The nucleic acid sequence may be introduced and expressed in any host organism, for example, in either prokaryotic or eukaryotic host cells. Examples of host cells include, without limitation, bacterial cells, yeast cells, cultured insect cell lines, and cultured mammalian cells lines. Preferably, the recombinant host cell system that is selected processes and post-translationally modifies nascent peptides in a manner desired to produce the immunogenic polypeptide or protein. In one embodiment, prokaryotic organisms may be utilized, for example, E. coli. In other embodiments, however, a eukaryotic host may be preferred, for instance the eukaryotic yeast P. pastoris.

The targeted LIP nucleic acid may be placed in expression cassettes for expression in the selected host. Such expression cassettes will comprise a transcriptional initiation region linked to the genetic sequence. Expression cassettes also may have a plurality of restriction sites for insertion of the nucleic acid to be under the transcriptional regulation of various control elements. The expression cassette additionally may contain selectable marker genes. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors may contain endogenous enhancers/promoters, splice junctions, intervening sequences, polyadenylation signals, etc., or a combination of both endogenous and exogenous control elements.

Preferably the nucleic acid in the vector is under the control of, and operably linked to, an appropriate promoter or other regulatory elements for transcription in a host cell. Generally, any suitable promoter may be used that is capable of operative linkage to the heterologous DNA such that transcription of the DNA may be initiated from the promoter by an RNA polymerase that may specifically recognize, bind to, and transcribe the DNA in reading frame. Moreover, while promoters of the present invention may include sequences to which an RNA polymerase binds, this is not a requirement of the invention. For example, promoters of the disclosed DNA constructs may include regions to which other regulatory proteins may bind in addition to regions involved in the control of the protein translation, including coding sequences.

The vector may, if desired, be a bi-functional expression vector that may function in multiple hosts. The transcriptional cassette generally includes in the 5′-3′ direction of transcription, a promoter, a transcriptional and translational initiation region, a DNA sequence of the targeted LIP, and a transcriptional and translational termination region functional in the organism. The termination region may be native with the transcriptional initiation region, may be native with the DNA sequence of the targeted LIP, or may be derived from another source.

Nucleic acids encoding entire LIP proteins or immunogenic segments thereof may be introduced into host cells by any method known to one of skill in the art. For example, such nucleic acids may be introduced into bacterial cells by commonly used transformation procedures such as by treatment with calcium chloride or by electroporation. If the polypeptides are to be expressed in eukaryotic host cells, nucleic acids encoding those peptides may be introduced into eukaryotic host cells by a number of means including calcium phosphate co-precipitation, spheroplast fusion, electroporation and so forth. When the eukaryotic host cell is a yeast cell, transformation may be affected by treatment of the host cells with lithium acetate or by electroporation.

A wide range of expression vectors is available in the art. Description of various expression vectors and how to use them may be found in, for example U.S. Pat. Nos. 5,604,118; 5,583,023; 5,432,082; 5,266,490; 5,063,158; 4,966,841; 4,806,472; and 4,801,537; and in Goedel et al., Gene Expression Technology, Methods of Enzymology, Vol. 185, Academic Press, San Diego (1989). Recombinant DNA and molecular cloning techniques that may be used to help make and use aspects of the invention are described by Sambrook et al., Molecular Cloning: A Laboratory Manual Vol. 1-3, Cold Spring Harbor laboratory, Cold Spring Harbor, N.Y. (2001); Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor laboratory, Cold Spring Harbor, N.Y. (1989); and by T. J. Silhavy, M. L. Berman, and L. W. Enquist, Experiments with Gene Fusions, Cold Spring Harbor Laboratory, Cold Spring Harbor, N.Y. (1984).

In one embodiment a prokaryotic E. coli expression system may be used. Useful E. coli vectors may contain constitutive or inducible promoters to direct expression of either fusion or non-fusion proteins. With fusion vectors, a number of amino acids are usually added to the expressed target gene sequence at the amino terminus. Additionally, a proteolytic cleavage site may be introduced at a site between the target recombinant protein and the fusion sequence. Once the fusion protein has been purified, the cleavage site allows the target recombinant protein to be separated from the fusion sequence. Enzymes suitable for use in cleaving the proteolytic cleavage site include Factor Xa and thrombin. Fusion expression vectors which may be useful in the present invention include pGex (Amrad Corp., Melbourne, Australia), pRIT5 (Pharmacia, Piscataway, N.J.) and pMAL (New England Biolabs, Beverly, Mass.), which fuse glutathione S-transferase, protein A, or maltose E binding protein, respectively, to the target recombinant protein.

Expression of unfused foreign genes in E. coli may be accomplished with recombinant vectors including, but not limited to, the E. coli expression vector pUR278 (Ruther et al., EMBO J. 2:1791, 1983). Using the pUR278 vector, the nucleotide sequence coding for the target gene product may be ligated in frame with the lac V coding region to produce a fusion protein. Other useful vectors include pin vectors (Inouye and Inouye, Nucleic Acids Res. 13:3101-3109, 1985) and so forth.

Expression of the targeted LIP gene may also be obtained using eukaryotic vectors such as mammalian, yeast or insect cells. The use of eukaryotic vectors permits partial or complete glycosylation and/or the formation of the relevant inter- or intra-chain disulfide bonds. Examples of vectors useful for expression in the yeast Saccharomyces cerevisiae include pYepSecI (Baldari et al. EMBO 6:229-234, 1987) and pYES2 (Invitrogen Corp., San Diego, Calif.).

Baculovirus vectors are also available for the expression of the proteins in cultured insect cells (F9 cells). The use of recombinant Baculovirus vectors may be, or is, analogous to the methods disclosed in “Baculovirus Expression Protocol”, ed. by C. D. Richardson, 1995, Humana Press Inc.; Smith et al., “Production of Human Beta Interferon in Insect Cells Infected with a Baculovirus Expression Vector,” Mol Cellular Biol 3:2156-2165, 1983; Pennock et al., Strong and Regulated Expression of Escherichia coli B-Galactosidase in Insect cells with a Baculovirus Vector, Mol Cellular Biol 4:399-406, 1984.

Other vectors useful for expressing the LIP proteins, or an epitope of the proteins, include viral vectors. Methods for making a viral recombinant vector useful for expressing the disclosed proteins are analogous to the methods disclosed in U.S. Pat. Nos. 4,603,112; 4,769,330; 5,174,993; 5,505,941; 5,338,683; 5,494,807; 4,722,848; E. Paoletti, “Applications of Poxvirus Vectors to Vaccination: An Update,” PNAS USA 93:11349-11353, 1996; Moss, “Genetically Engineered Poxviruses for Recombinant Gene Expression, Vaccination and Safety,” PNAS USA 93:11341-11348, 1996; Roizman, “The Function of Herpes Simplex Virus Genes: A Primer for Genetic Engineering of Novel Vectors,” PNAS USA 93:11307-11302, 1996; Frolov at al., “Alphavirus-Based Expression Vectors: Strategies and Applications,” PNAS USA 93:11371-11377, 1996; Grunhaus et al., “Adenoviruses As Cloning Vectors,” Seminars in Virology 3: 237-252, 1993 and U.S. Pat. Nos. 5,591,639; 5,589,466; and 5,580,859, relating to DNA expression vectors.

In accordance with one embodiment of the present invention, isolated and/or purified antibodies that recognized and bind LIP proteins may be generated for inclusion in a diagnostic device as herein described. For instance, according to one embodiment, substantially pure recombinant polypeptide suitable for use as an immunogen may be isolated from cells in which it is produced and then polyclonal antiserum containing antibodies to heterogeneous epitopes of an LIP protein may be prepared by immunizing suitable hosts with the expressed polypeptide, which may be unmodified or modified to enhance immunogenicity. As is generally known in the art, effective polyclonal antibody production may be affected by many factors related both to the antigen and the host species. For example, small molecules tend to be less immunogenic than others and may require the use of carriers and adjuvant. Also, host animals may vary in response to site of inoculations and dose, with both inadequate and excessive doses of antigen resulting in low titer antisera.

Booster injections may be given at regular intervals, and antiserum harvested when antibody titer thereof, as determined semi-quantitatively, for example, by double immunodiffusion in agar against known concentrations of the antigen, begins to fall. See, for example, Ouchterlony et al. (Handbook of Experimental Immunology, Wier, D. (ed.) chapter 19. Blackwell (1973)). In general, plateau concentration of antibody may usually be in the range of 0.1 to 0.2 mg/ml of serum. Affinity of the antisera for the antigen is determined by preparing competitive binding curves, as described, for example, by Fisher (Manual of Clinical Immunology, Ch. 42. (1980)).

Another possible approach to raising antibodies against the LIP proteins may utilize synthetic peptides synthesized on a commercially available peptide synthesizer based upon the amino acid sequence correlating to the known LIP gene sequences.

Antibodies may optionally be raised against the LIP proteins by subcutaneous injection of a DNA vector that expresses the polypeptide into laboratory animals, such as mice. Delivery of the recombinant vector into the animals may be achieved according to methods as are generally known in the art.

In another embodiment, monoclonal antibodies may be raised by hybridoma cells, phage display libraries, or other methodology. Monoclonal antibodies may be e.g., human, rat, or mouse derived. For the production of human monoclonal antibodies, hybridoma cells may be prepared by fusing spleen cells from an immunized animal, e.g., a mouse, with a tumour cell. Appropriately secreting hybridoma cells may thereafter be selected according to, for example, the method of Kohler and Milstein (Nature 256:495 (1975)), or derivative methods thereof. (Procedures for monoclonal antibody production are also described in Harlow and Lane (1988). Antibodies, A Laboratory Manual, Cold Spring Harbor Laboratory, New York; Cole, et al., “Monoclonal antibodies and Cancer Therapy, Alan R. Liss, Inc., pp. 77-96.). Rodent antibodies may be humanised using recombinant DNA technology according to techniques known in the art. Alternatively, chimeric antibodies, single chain antibodies, Fab fragments, and so forth may also be developed against the LIP polypeptides using skills known in the art.

The present invention may be better understood with reference to the following examples.

### Experimental Procedures

ELISA Assay

Polyclonal antibodies against recombinant LIP5 (residues 57-403 with a C-terminal LEHHHHHH peptide fusion), purified by Protein A affinity chromatography, were screened by Enzyme Linked Immunosorbant Assay (ELISA) as follows: Purified protein was coated at 2 micrograms per milliliter (μg/ml) with 50 microliters (μL) per well on a 96 well flat bottomed plate for 1 hour at room temperature. Wells were then blocked in 200 μL of 1×PBS/1% BSA solution for 1-2 hours and briefly rinsed in 200 μL 1×PBS/0.02% NaN₃. Antibody samples and a negative control were serial diluted in a 5 fold series beginning at 1/50 in 1×PBS/1% BSA in duplicate and incubated for 1 hour. An HRP secondary antibody was diluted at 1/2000 in 1×PBS/1% BSA and incubated for 1 hour. Both primary and secondary antibody incubations were followed by 3 washes in 200 μl of 1×PBS/0.05% Tween 20. Color signals were developed with 50 μL of TMB solution followed within 2 to 5 minutes by 50 μL TMB stop solution. Plates were read on a spectrophotometer at 450 nanometers (nm).

IgG Purification

Antibodies to LIP5 (residues 57-403 with a C-terminal LEHHHHHH peptide fusion) were enriched from 6 milliliters (ml) of crude sample using a 1 ml protein A agarose column. The column was attached to a peristaltic pump and the flow through was diverted through an optical reader. Crude sample was diluted in binding buffer to a total volume of 16 ml and loaded onto the column in 5 ml increments. After loading, the column was washed in 15 ml binding buffer, followed by 5 ml of elution buffer. The final eluate had a volume of 2 ml at its greatest peak. This process was repeated two times for final combined eluate volume of 6 ml. The eluate was then exchanged into 1×PBS buffer by spinning through an Amicon Ultra 15 30 kDa cutoff centrifuge filter at 4500×g.

Western Blotting

Protein samples were diluted in 1× sample buffer with 0.1 M DTT and run on a 4-12% SDS-PAGE gel for 45 minutes at a constant 200V. Protein was then transferred from the acrylamide gel to PVDF membrane for 1 hour at a constant 30V. The membrane was blocked in 10 ml block solution of 2% BSA in 1×PBS/0.2% Tween for 1 hour at room temperature or overnight at 4° C. Purified polyclonal antibody was used at a dilution of 1/5000 in 10 ml of primary antibody dilution buffer and incubated with shaking for 1 hour at room temperature. The membrane was then washed three times in 1×PBS/0.2% Tween for 5 minutes each and then transferred to 10 ml of secondary antibody solution for 30 minutes while shaking at room temperature provided by the WesternBreeze Chromogenic Kit from Invitrogen. The membrane was washed again three times in 1×PBS/0.2% Tween for 5 minutes each. The signal was developed via incubation with 5 ml of chromogenic substrate specific for sample antibodies for 5 to 30 minutes (depending upon the desired strength of signal), followed by a final rinse in ddH₂O, and air drying overnight.

### EXAMPLE 1

Genomic DNA from Candida albicans culture (ATCC strain 10231 D) was isolated and the target LIP5 gene was amplified via PCR. Recombinant LIP5 (residues 57-403) was successfully subcloned into an E. coli expression vector pET28 (available from Novagen) and purified in 6M urea using an FPLC via a c-terminal 6× histidine tag. The protein was refolded via dialysis in 1×PBS; final yields of 10-20 mg of protein (38.4 kDa) were obtained from 1 liter of bacterial culture.

IgGs were generated. Following generation they were purified using a protein A agarose column. This increased the effective concentration of the polyclonal antibodies.

The purified antibodies were probed against recombinant LIP5 protein and also against LIP4 protein and results were measured by ELISA. Results are illustrated in FIG. 3. As may be seen with reference to the figure, the observed signals were found to be identical within the error of the assay. This indicates that the anti-LIP5 polyclonal antibody may be used to detect both LIP5 protein and LIP4 protein. As LIP8 protein is even more homologous to LIP5 than is LIP4, it is believed that this cross-reactivity will function between these materials as well.

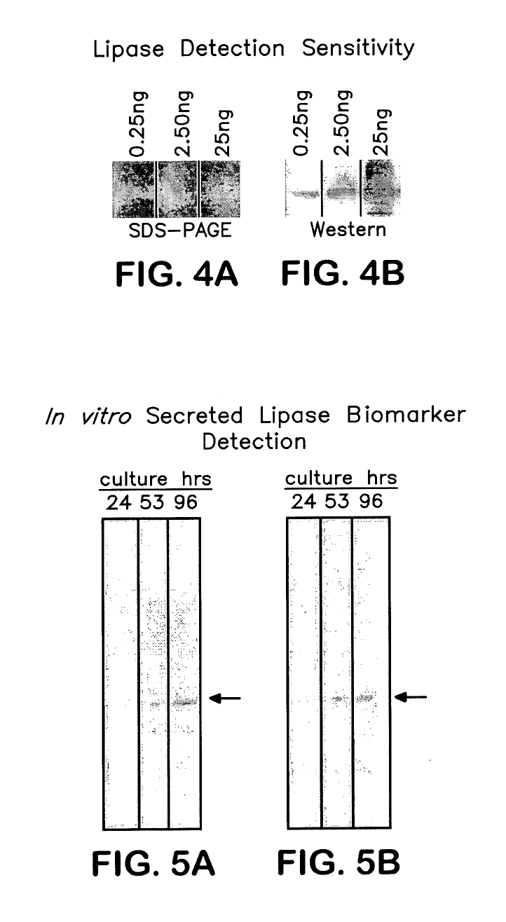

The limit of detection of recombinant LIP5 protein by the anti-LIP5 antibodies was determined by western blot (FIG. 4B). A PVDF membrane was coated with purified LIP5 protein in each of three strips at decreasing amounts of 25 ng, 2.50 ng, and 0.25 ng, as shown. Each strip was probed separately with the anti-LIP5 polyclonal antibody. As may be seen with reference to FIG. 4B, the recombinant lipase signal may clearly be seen down to 0.25 ng. In contrast, 25 ng is required to observe a signal by standard SDS-PAGE (FIG. 4A). The 0.25 ng that may be detected by western blot converts to about 33 ng/ml of LIP5 protein in the primary sample (for instance, in a culture supernatant).

### EXAMPLE 2

Cultures of C. albicans (ATCC strain #10231) were grown in rich media (YPD) at 37° C. for long incubation times as indicated on FIG. 5A. the anti-LIP5 antibodies described in Example 1 were used to detect a signal with the expected molecular weight of native secreted lipase (indicated on the figure by the arrow). The signal corresponds to an apparent molecular weight of ˜55 kDa; this is in good agreement the predicted molecular weight of ˜50 kDa plus potential glycosylation at the known internal glycosylation site.

FIG. 5B illustrates the results when the cultures were grown in minimal media (YNB), supplemented with 2.5% Tween20 as a sole carbon source, at 37° C. for the indicated times. Again, a signal develops of the appropriate molecular weight that is specifically recognized by anti-LIP5 antibodies.

These observations demonstrate that anti-LIP antibodies may be used to detect secreted lipases from model disease systems and that the antibodies may be used to distinguish between normal culture conditions and the model disease system.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A diagnostic test kit for detecting a lipase protein within a test sample, the diagnostic test kit comprising:
an assay device comprising a fluidic medium, the fluidic medium defining a detection zone within which is immobilized a receptive material, wherein the detection zone is capable of generating a detection signal that corresponds to the presence or absence of a first lipase protein; and
a detection probe conjugated with a binding member;
wherein the receptive material, the binding member, or both contain a first antibody that is capable of specifically binding to the first lipase protein.

2. The diagnostic test kit of claim 1, wherein the receptive material contains the first antibody.

3. The diagnostic test kit of claim 2, wherein the binding member contains a second antibody that specifically binds to the first lipase protein.

4. The diagnostic test kit of claim 3, wherein the first antibody is a monoclonal antibody that specifically binds to a first epitope of the first lipase protein and the second antibody is a monoclonal antibody that specifically binds to a second epitope of the first lipase protein.

5. The diagnostic test kit of claim 1, wherein the binding member contains the first antibody.

6. The diagnostic test kit of claim 1, wherein the first antibody is a monoclonal antibody.

7. The diagnostic test kit of claim 1, wherein the first antibody is a polyclonal antibody.

8. The diagnostic test kit of claim 7, wherein the polyclonal antibody has been developed against a second lipase protein or an immunogenic fragment thereof.

9. The diagnostic test kit of claim 8, wherein the second lipase protein has more than about 73% sequence identity with the first lipase protein.

10. The diagnostic test kit of claim 1, wherein the fluidic medium is a porous membrane.

11. The diagnostic test kit of claim 1, wherein the antibody has been developed against a recombinant lipase protein or an immunogenic fragment thereof.

12. The diagnostic test kit of claim 11, wherein the first antibody has been developed by a hybridoma cell that has been generated through fusion of a human tumor cell with a cell from an animal immunized with the recombinant lipase protein or an immunogenic fragment thereof.

13. The diagnostic test kit of claim 1, wherein the diagnostic test kit is capable of detecting infection by the pathogenic form of Candida albicans.

14. A diagnostic test kit for detecting a lipase protein within a test sample, the diagnostic test kit comprising:
an assay device comprising a porous membrane, the porous membrane defining a detection zone within which is immobilized a first antibody, wherein the detection zone is capable of generating a detection signal that corresponds to the presence or absence of a first lipase protein; and
a detection probe conjugated with a second antibody;
wherein the first antibody and the second antibody are capable of specifically binding to the first lipase protein.

15. The diagnostic test kit of claim 14, wherein the first antibody is a monoclonal antibody that specifically binds to a first epitope of the first lipase protein and the second antibody is a monoclonal antibody that specifically binds to a second epitope of the first lipase protein.

16. The diagnostic test kit of claim 14, wherein the first antibody, the second antibody, or both are monoclonal antibodies.

17. The diagnostic test kit of claim 14, wherein the first antibody, the second antibody, or both are polyclonal antibodies.

18. The diagnostic test kit of claim 17, wherein the first antibody, the second antibody, or both have been raised against a second lipase protein or an immunogenic fragment thereof.

19. The diagnostic test kit of claim 18, wherein the second lipase protein has more than about 73% sequence identity with the first lipase protein.

20. The diagnostic test kit of claim 14, wherein the first antibody, the second antibody, or both are developed against a recombinant lipase protein or an immunogenic fragment thereof.

21. The diagnostic test kit of claim 20, wherein the first antibody, the second antibody, or both are developed by a hybridoma cell that has been generated through fusion of a human tumor cell with a cell from an animal immunized with the recombinant lipase protein or an immunogenic fragment thereof.

22. A method for detecting the presence of a lipase protein within a test sample, said method comprising:
i) providing an assay device that comprises a fluidic medium, the fluidic medium defining a detection zone within which is immobilized a receptive material, the detection zone being in fluid communication with detection probes conjugated with a binding member, wherein the receptive material, the binding member, or both contain a first antibody that is capable of specifically binding to a first lipase protein;
ii) contacting the assay device with the test sample; and
iii) generating a detection signal within the detection zone that corresponds to the presence or absence of the first lipase protein.

23. The method of claim 22, wherein the receptive material contains the first antibody.

24. The method of claim 22, wherein the binding member contains a second antibody that specifically binds to the first lipase protein.

25. The method of claim 22, wherein the binding member contains the first antibody.

26. The method of claim 22, wherein the first antibody is a monoclonal antibody.

27. The method of claim 22, wherein the first antibody is a polyclonal antibody.

28. The method of claim 27, wherein the first antibody has been raised against a second lipase protein or an immunogenic fragment thereof.

29. The method of claim 28, wherein the second lipase protein has more than about 73% sequence identity with the first lipase protein.

30. The method of claim 22, wherein the first antibody is developed against a recombinant lipase protein or an immunogenic fragment thereof.

31. The method of claim 30, wherein the first antibody is formed by a hybridoma cell that has been generated through fusion of a human tumor cell with a cell from an animal immunized with the recombinant lipase protein or an immunogenic fragment thereof.

32. The method of claim 22, further comprising measuring the intensity of the detection signal, wherein the amount of the lipase in the test sample is proportional to the intensity of the detection signal.

33. The method of claim 22, further comprising obtaining the test sample from a host, wherein the detection signal indicates the presence of a pathogen in the host, wherein the pathogen expresses lipase proteins.

34. The method of claim 33, wherein the pathogen is the pathogenic form of Candida albicans.

35. The method of claim 22, wherein the test sample is obtained from vaginal fluid.

36. The method of claim 22, wherein the test sample is obtained from saliva.

37. The method of claim 22, wherein the test sample is obtained from blood.

38. The method of claim 22, wherein the test sample is obtained from mammalian tissue.

39. The method of claim 38, wherein the mammalian tissue is skin.
